(19) United States
(12) Patent Application Publication
(10) Pub. No.: US 2003/0087298 A1
(43) Pub. Date: May 8, 2003

(54) Detection of hybridization on oligonucleotide microarray through covalently labeling microarray probe

(76) Inventors: Roland Green, Madison, WI (US); Thomas Albert, Madison, WI (US)

Correspondence Address: Nicholas J. Seay, Quarles & Brady LLP, P O Box 2331, Madison, WI, 53701-2113, US

(21) Appl. No.: 10/287,722

(22) Filed: Nov. 1, 2002

Related U.S. Application Data
(60) Provisional application No. 60/335,377, filed on Nov. 2, 2001.

(51) Int. Cl. C12Q 1/68; C12P 19/34

(52) U.S. Cl. 435/6; 435/91.2

(57) ABSTRACT

A method for detecting hybridization between a probe with a free 3′ end on an oligonucleotide microarray and a polynucleotide from a sample is disclosed. The method involves contacting the sample with the probe under a hybridization condition under which desired hybridization events occur, performing an elongation reaction on the probe using at least one labeled nucleotide, removing unincorporated labeled nucleotides from the microarray, and determining whether the probe is labeled.

### CROSS-REFERENCE TO RELATED APPLICATIONS

This application claims benefit from U.S. Patent Application No. 60/335,377 filed Nov. 2, 2001.

### STATEMENT REGARDING FEDERALLY SPONSORED RESEARCH OR DEVELOPMENT

Not applicable.

### BACKGROUND OF THE INVENTION

The advent of DNA microarray technology makes it possible to build an array of hundreds of thousands of DNA sequences in a very small area, such as the size of a microscopic slide. See, e.g., U.S. Pat. No. 6,375,903 and U.S. Pat. No. 5,143,854, each of which is hereby incorporated by reference in its entirety. The disclosure of U.S. Pat. No. 6,375,903 enables the construction of so-called maskless array synthesizer (MAS) instruments in which light is used to direct synthesis of the DNA sequences, the light direction being performed using a digital micromirror device (DMD). Using an MAS instrument, the selection of DNA sequences to be constructed in the microarray is under software control so that individually customized arrays can be built to order. In general, MAS based DNA microarray synthesis technology allows for the parallel synthesis of over 800,000 unique oligonucleotides in a very small area of on a standard microscope slide. The microarrays are generally synthesized by using light to direct which oligonucleotides are synthesized at specific locations on an array, these locations being called features.

Microarrays are most often used to conduct hybridization procedures with nucleic acids of unknown character. Hybridization using DNA microarrays, both between DNA and DNA, and DNA and RNA, has been used widely in many different applications such as toxicity testing, genetic testing and disease gene detection. However, the most common application for which the use of microarrays is popular is for gene expression studies. For the typical gene expression study, whole RNA is extracted from a cell or tissue and then used in a hybridization procedure against the microarray. The gene which are expressed in the cell or tissue are detected by virtue of the mRNA species which hybridize against the microarray DNA probes. Gene expression studies are used to determine gene function, to study the developmental biology of organisms, to study the processes of disease and for any number of other applications of scientific and medical interest.

The conventional method to prepare a labeled polynucleotide sample for hybridization with an oligonucleotide microarray involves isolating RNAs from a source, and then labeling the RNA with a marker molecule. The marker molecule is typically a fluorescent marker which is covalently attached to the RNA in the experimental sample. This conventional method is inconvenient and has several disadvantages. First of all, the cost of labor and reagents in preparing the RNA sample for hybridization, i.e. the process of adding the marker, is relatively high. The process of sample preparation also requires time and the use of skilled labor. It would be advantageous if the sample preparation process could be simplified.

### BRIEF SUMMARY OF THE INVENTION

The present invention provides a method for detecting hybridization between a DNA probe with a free 3′ end on an oligonucleotide microarray and a RNA polynucleotide from a sample. The method involves contacting the RNA sample with the probe under a hybridization condition under which desired hybridization events occur, performing an elongation reaction on the probe using at least one labeled nucleotide, removing unincorporated labeled nucleotides from presence of the microarray, and determining whether the probe is labeled.

One advantage of the present invention is that highly stringent washing conditions can be used to wash off the unincorporated labeled nucleotides at the end of an elongation reaction to reduce background signal and hence improve assay sensitivity. The reason that the method of the present invention can withstand highly stringent washing conditions is because the label is covalently attached to the probes.

Another advantage of the present invention is that it significantly simplifies polynucleotide sample preparation prior to hybridization reaction. Only a simple DNA or RNA extraction step is necessary to obtain a DNA or RNA sample for hybridization.

Still another advantage of the present invention is that it eliminates the labeling problem for bacterial samples which do not have polyA tail.

A further advantage of the present invention is that it eliminates the reverse transcription and transcription steps in the conventional method which can introduce bias into the analysis.

### DETAILED DESCRIPTION OF THE INVENTION

The present invention provides a method for performing direct hybridization between a DNA probe on a oligonucleotide microarray that has a free 3′ end and an unlabeled polynucleotide, such as RNA, in a sample. The method involves contacting the sample with the probe under a hybridization condition under which desired hybridization events occur, performing an elongation reaction on the probe to incorporate at least one labeled nucleotide into the hybridized complex, removing unincorporated labeled nucleotides from the microarray, and determining whether the probe is labeled. Optionally, one or more washing steps can be performed after the unincorporated labeled nucleotide has been removed. If there is a successful hybridization between a probe and a polynucleotide in the sample, there will a probe-polynucleotide duplex with the polynucleotide overhanging the probe's 3′ end. The elongation will add labeled nucleotide to the probe so that the probe is labeled. The label in the complex can then be detected to determine where hybridization has occurred.

Since the detection method of the present invention depends the addition of one or more labeled nucleotides to the DNA probes on the microarray, the DNA probes have to have their 3′ ends free for such addition, so that the normal 5′ to 3′ DNA extension reaction can be performed. In the past, microarrays have mainly been constructed 3′ to 5′, or in the reverse direction from normal biological DNA sythesis, because of the needs of the photo-labile chemistry used. It has been found that a class of photo-labile protecting groups, known as NPPOC, can readily be adapted for used in the 5′ to 3′ orientation. This chemistry is described in U.S. Pat. Nos. 5,763,599 and 6,153,744, to Pfleiderer et al., the disclosures of which are hereby incorporated by reference. The only significant difference in the use of this chemistry is that the photo-labile groups is attached to the 3′ end of each nucleotide rather than the 5′ end. Other chemistries are also known which can be used to make microarrays with free 3′ ends, see for example U.S. Pat. No. 5,908,926 to Pirrung. All this is important for the microarray is that it have free 3′ ends on the DNA probes.

The sample nucleic acids are added to the microarray without being labeled beforehand. Assuming a normal gene expression study is being performed, whole mRNA can be simply extracted from a cell or tissue and used. The probes are selected so that the mRNA species which hybridize to the probe extend beyond the end of the probe. Then a template dependent DNA extension reaction is performed to add nucleotides to the probe DNA if and only if a hybridized mRNA is present. One of ordinary skill in the art knows how to carry out elogation reactions, typically using a reverse transcriptase. It is well within the knowledge of one of ordinary skill in the art to select the right enzyme and nucleotides for an elongation reaction depending on whether the polynucleotides in the sample are DNA or RNA. The hybridization and the elongation reactions can be performed at the same time or the elongation reactions can be performed after the hybridization reactions. One of ordinary skill in that art knows how to do each. After the elongation reaction is performed, one or more washing steps are preferably performed to remove unbound labeled nucleotides. Other washings may be appropriate in between for helping control hybridization stringency and making subsequent hybridization reactions more efficient.

Labeled nucleotides used for elongation reactions can be labeled in different ways as long as the detection tool engaged can detect the label. For example, the nucleotides can be labeled by fluorescent material, radioactive material or other detectable agents. Most oligonucleotide microarrays are scanned by fluorescence scanners and thus the nucleotides are labeled to fluoresce. There are many compounds and methods that one can label a nucleotide with fluorescence. One of ordinary skill in the art is familiar with these compounds and methods. Methods of detection for labels other than fluorescence are also familiar to one of ordinary skill in the art. Under most circumstances, all four types of nucleotides (A, T, G and C for DNA, and A, U, G and C for RNA) are used for an elongation reaction. At least one and preferably more than one type of nucleotides used for an elongation reaction are labeled.

The stringency of the hybridization reaction conditions used in the present invention should be adjusted according to factors in individual applications such as the length of the probes, the expected length of complement sequences, the number of mismatches that can be allowed. One of ordinary skill in the art knows how to determine hybridization conditions to allow desired hybridizations occur while limiting non-specific hybridizations. We here only provides an example as to how one of ordinary skill in the art can control hybridization stringency through hybridizations and washing conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE).

Then, assuming that 1% mismatching results in a 1° C. decrease in the Tm, the temperature of the final wash in the hybridization reaction is reduced accordingly. In practice, the change in Tm can be between 0.5° C. and 1.5° C. per 1% mismatch. Stringent conditions involve hybridizing at 68° C. in 5×SSC/5× Denhardt's solution/1.0% SDS at room temperature. Moderately stringent conditions include washing in 3×SSC at 42° C. The parameters of salt concentration and temperature can be varied to achieve the optimal level of identity between the probe and the target nucleic acid. Additional guidance regarding such conditions is readily available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) At Unit 2.10.

One advantage of the present invention is that highly stringent washing conditions can be used to wash off the unincorporated labeled nucleotides at the end of an elongation reaction to reduce background signal and hence improve assay sensitivity. The reason that the method of the present invention can withstand highly stringent washing conditions is because the label is covalently attached to the probes.

### EXAMPLE

To demonstrate the ability of a primer extension process to be performed on a DNA microarray in situ, a DNA microarray was constructed using a maskless array synthesizer of the type described in U.S. Pat. No. 6,375,903. The DNA probes in the microarray were 24 nucleotides in length. The probes were constructed in the 5′ to 3′ orientation using special phosphoamidites synthesized with the NPPOC photo-labile protecting groups added to the 3′ end of each nucleoside. The probe sets were constructed with each test probes for the sequence to be assayed in the sample also having a single base mis-match probes also constructed elsewhere in the array, with the single mis-match being located at the 3′ end of each probe.

The probes were designed to test for the presence or absence of one of two alleles of the human ABO blood type gene. The experimental sample was cRNA made from whole mRNA extracted from human blood from several donors. No RNA in the sample was labeled at all. The experimental sample was applied to the microarray and allowed to hybridize. Then Cy3 labeled nucleosides were added to the reaction chamber along with MMLV reverse transcriptase, to perform a DNA extension reaction adding nucleotides to those probes to which an RNA strand had hybridized. The ends of the probes, and the single base mismatches, were at nucleotide position 261 of the human ABO gene. The microarray was read for presence or absence of fluorescence in each features using conventional fluorescent scanning techniques.

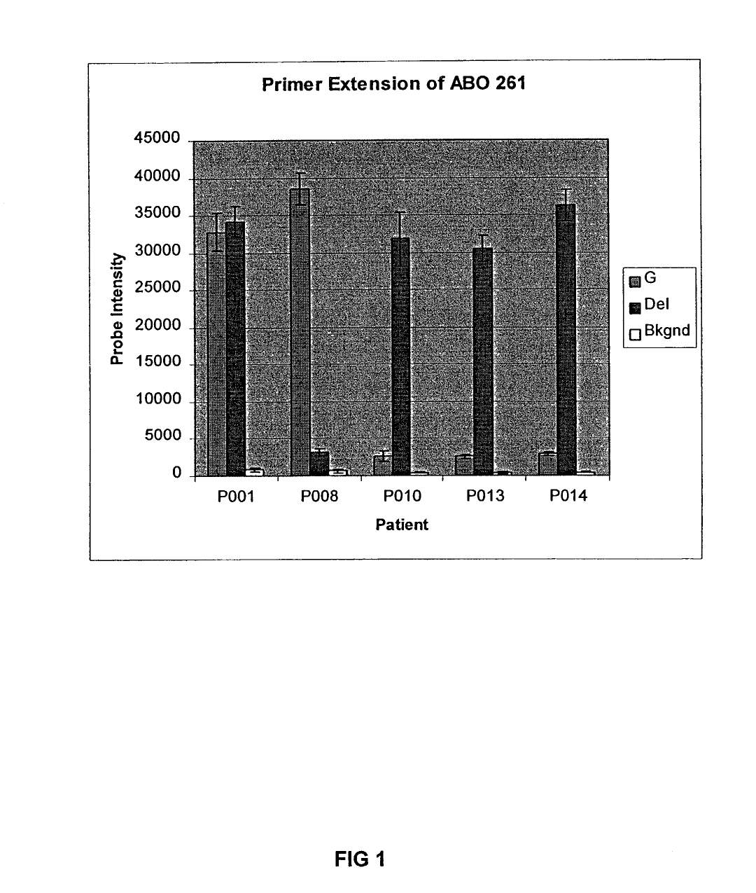

The results of this example are presented in FIG. 1. The sample designated P001 was from an individual having both the A and B alleles. Similarly, the sample designated P008 was from a patient known to have only one allele and patients P010, P013 and P014 had only the other allele. Non-specific fluorescence was no higher than background and no fluorescence was detected at alleles not present in the patients. This demonstrates that probe extension reactions occurred only where hybridization occurred.

I/We claim:

1. A method for detecting hybridization between a DNA probe on a microarray and a polynucleotide from a sample, the method comprising the steps of:
(a) providing a microarray with DNA probes having free 3′ ends;
(b) contacting the sample with the microarray under a hybridization conditions under which desired hybridization events occur;
(c) performing a template-dependent elongation reaction on the microarray in the presence of at least one labeled nucleotide;
(d) removing unincorporated labeled nucleotides from the microarray; and
(e) determining whether the probes in the microarray have been labeled.

2. The method of claim 1, wherein the probe is DNA.

3. The method of claim 1, wherein the polynucleotide is DNA.

4. The method of claim 1, wherein the polynucleotide is RNA.

5. The method of claim 1 further comprising the step of washing the oligonucleotide microarray.

6. A method for performing an assay of gene expression in a cell or tissue, the method comprising the steps of:
(a) providing a microarray with DNA probes having free 3′ ends and with probes designed to detect the expression of genes that might be expressed in the cell or tissue;
(b) extracting RNA from the cell or tissue;
(c) contacting the extracted RNA with the microarray under a hybridization conditions under which desired hybridization events occur without labeling the extracted RNA;
(d) performing a template-dependent elongation reaction on the microarray in the presence of at least one labeled nucleotide;
(e) removing unincorporated labeled nucleotides from the microarray; and
(f) determining whether the probes in the microarray have been labeled.

7. The method of claim 6, wherein the probe is DNA.

8. The method of claim 6, wherein the polynucleotide is DNA.

9. The method of claim 6, wherein the polynucleotide is RNA.

10. The method of claim 6 further comprising the step of washing the oligonucleotide microarray.
